(19) United States
(12) Patent Application Publication
(10) Pub. No.: US 2015/0030502 A1
(43) Pub. Date: Jan. 29, 2015

(54) ARRANGEMENT WITH A BLOOD PUMP AND PUMP CONTROL UNIT

(71) Applicant: NOVALUNG GMBH, Heilbronn (DE)

(72) Inventors: Holger GORHAN, Schoenaich (DE); Georg MATHEIS, Heilbronn (DE)

(73) Assignee: NOVALUNG GMBH, Heilbronn (DE)

(21) Appl. No.: 14/444,248

(22) Filed: Jul. 28, 2014

(30) Foreign Application Priority Data
Jul. 29, 2013 (DE) 10 2013 012 433.6

(51) Int. Cl. A61M 1/10; A61M 1/16; A61M 1/26

(52) U.S. Cl. CPC A61M 1/1006; A61M 1/101; A61M 1/267; A61M 1/1698; A61M 2205/3341; A61M 2230/04; A61M 2230/30
USPC 422/45; 604/151; 604/66

(57) ABSTRACT

An arrangement for extracorporeal life support is further developed in such a way that a pump actuating signal produces a wave-like surging and subsiding pump output for a pulsatile flow. The pump is preferably a non-occlusive blood pump, such as a diagonal pump, for example. In a preferred variant of embodiment the control signal is provided by an ECG. This allows the diastolic pressure to be increased in order to improve the oxygen balance of the heart muscle.

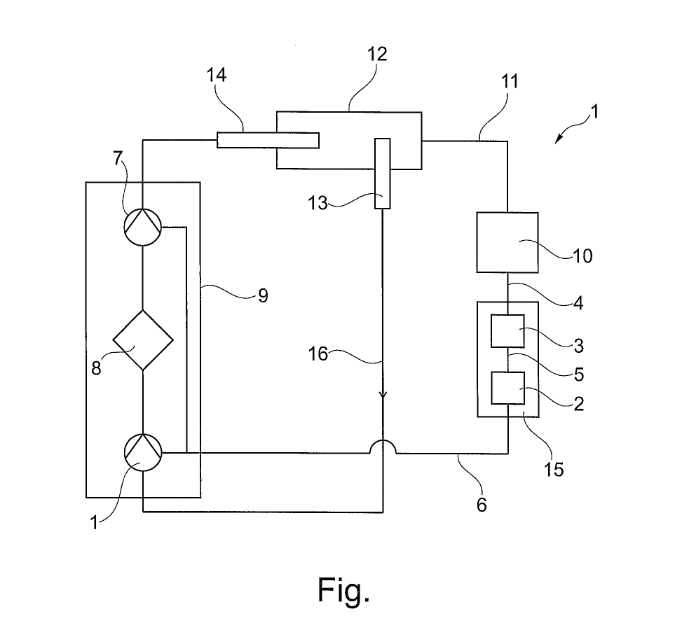

The blood pump relates to an arrangement with a blood pump and a pump control unit which has a computer that converts a control signal into a pump actuating signal.

Such arrangements are used for extracorporeal life support (ECLS) for example.

ECLS is used, for example, in patients with cardiogenic shock or decompensated heart failure, whose heart is no longer able to supply the body sufficiently with oxygen-rich blood.

The purpose of the invention is to further develop such an arrangement and to propose a method of operating a blood pump.

This objective is achieved with an arrangement of the type in question in which the pump actuating signal brings about a wave-like surging and subsiding pump output for a pulsatile flow. The pulsatile flow produced by the pump actuating signal improves the circulatory situation.

A wave-like surging and subsiding pump output does not mean a constant pump stroke or switching the pump on and off, but a pump output that is produced by a variable control signal and varies over time.

The arrangement makes a cardiac support system possible that emits pulses integrated into the cardiac cycle in order to improve the blood supply to the coronary vessels and better supply the heart with oxygen.

It is advantageous if the blood pump also provides a constant basic output. In this way the systemic perfusion pressure is increased with a laminar base flow.

This constant basic output can be provided by the pump which also brings about the pulsatile flow. Depending on the area of application it may be advantageous for the arrangement to have a further blood pump which provides the constant basic output.

In this case the further pump can also provide a wave-like surging and subsiding pump output.

In this way the pulsatile flow and the constant basic output can be provided either by means of one pump or the surging and subsiding pump output and constant basic output functions are split between two pumps.

However, two pumps can also be used which each provide a wave-like surging and subsiding pump output. With a second pump time operating in a time-delayed manner with regard to the first blood pump, it is possible to provide a wave-like surging and subsiding pump output so that the pressures waves overlap.

Such an arrangement usually has an oxygenator which is supplied by the pump. In principle the pump can be arranged either upstream or downstream of the oxygenator. It is of advantage if one blood pump is arranged upstream of the oxygenator in the direction of flow and a further blood pump is arranged downstream of the oxygenator.

A preferred variant of embodiment envisages that the oxygenator has a housing and that at least one blood pump is arranged in this housing. This makes it possible to arrange, for example, a blood pump in the housing of the oxygenators upstream of the oxygenator or downstream of the oxygenator.

A particularly advantageous variant of embodiment envisages that the arrangement has at least one non-occlusive blood pump, such as, in particular, a diagonal, axial or centrifugal pump.

In order to provide the required control signal it is envisaged that the arrangement has a clock generator. In accordance with a predetermined rhythm, this clock generator can provide the control signal for the pump in terms of frequency and amplitude. In this way the wave-like surging and subsiding pump output is achieved.

In a particularly preferred variant of embodiment this control signal is provided by an ECG. For this, software with the ability to record an ECG signal is integrated into the control unit of an ECLS system. A patient cable derives the ECG signal on the patient. Preferably the thus recorded R wave is the clock generator (trigger) for emitting a software trigger for starting the blood pump which then generates the pulse. The software ensures the precise emission of the pulse to the cardiac cycle, preferably the diastole. Advantageously it is ensured that the duration of the pulse is adapted in such a way that at the start of systole the pulse is no longer present. However, a pulse profile can also be generated which acts on the systole and/or on the diastole.

Cumulatively or alternatively it is proposed that the arrangement has an arterial pressure sensor which provides the control signal. This makes it possible to influence the pump output by means of a pressure measurement on an artery.

Experience has shown that it is advantageous if the arrangement has an arterial cannula which is longer than around 20 cm, preferably longer than 30 cm. The particularly long cannula serves to ensure that the pulse is emitted as closely to the heart as physiologically possible.

The aim on this the invention is based is also achieved with a method for operating a blood pump, in which the pump is operated with an iterating output in order to produce a wave-like surging and subsiding pulsatile flow.

Phase-shifted in relation to the pulsatile flow, a further blood pump can bring about a wave-like surging and subsiding pump output.

It is advantageous if the pulsatile flow of at least one pump is overlapped by a base load.

In the implementation of the procedure it is preferably ensured that the diastolic pressure is increased with the pump. This allows the circulation support to be produced with an ECLS system in such a way that in addition to a laminar base flow the pulsatile function is adjusted so that a flow and pressure increase takes places in the diastole phase. Triggering of the system preferably takes place through synchronisation with the heart.

The described arrangement can, however, also be used to direct the flow to an oxygenator with the pump. The pulsatility improves the function and service life of the oxygenator.

An example of such an arrangement is shown in the drawing and will be described in more detail below. The single figure schematically shows the individual elements of the arrangement and their interconnection.

Essential elements of the arrangement 1 are a first blood pump 1, a pump control unit 2 and a computer 3. The computer 3 converts a control signal 4 into a pump actuating signal. Via the pump control unit 2 this pump actuating signal 5 produces a wave-like surging and subsiding pump output on the pump 1 which thereby brings about a pulsatile flow.

Via the lead 6, the pump control unit 2 is connected to the first pump 1 and a further pump 7. This makes it possible to produce both basic load and also pulsatile flow with the first pump 1 which is arranged upstream of an oxygenator 8. However, with the first pump 1 upstream of the oxygenator 8 a basic load can also be produced, and with the second pump 7 downstream of the oxygenator 8 a pulsatile flow.

Finally, in each case a pulsatile flow can also be achieved with the first pump 1 upstream of the oxygenator 8 and the second pump 7 downstream of the oxygenator. Because of the distance between the pumps, this makes it possible to overlap time-delayed waves or to control the pumps with time-delayed signals.

Together with the oxygenator 8, the pumps 1 and 7 are arranged in a housing 9. This permits a simple construction. In the shown example of embodiment only one lead 6 runs from the pump control unit 2 to the housing 9 in order in the housing 9 to provide the two pumps 1 and 7 with a pump actuating signal. As an alternative one lead can be taken to the first pump 1 and a further lead to the second pump 7.

As a blood pump a diagonal pump is used, at least for the first pump 1. Preferably both pumps 1 and 7 are diagonal pumps. However, axial or centrifugal pumps can also be used.

The control signal 4 is provided by an ECG 10 which is connected to the patient 12 via a cable 11.

Located in the blood circulation or heart of the patient 12 are a venous cannula 13 and an arterial cannula 14. The arterial cannula is around 35-40 cm, preferably 30 to 45 cm, in length and the venous cannula is introduced into the vena cava.

During operation of the ECLS system, with the ECG 10, via the lead 11 an ECG signal of a patient 12 is recorded in order to generate a control signal 4. This control signal 4 is converted by the computer 3 into a pump signal 5 which, via the pump control unit 2 and lead 6 controls the pumps 1 and 7 or provides them with a current. A console 15 is used which emits a software trigger to start the blood pump 1 in accordance with a specially developed algorithm with the aim of emitting impulses into the systole and/or the diastole.

For this the ECG signal is implemented in the console. The user interface is adapted in order to create settings options for the ECG and to constitute a marker channel to show the relevant action of the blood pump as a sense or pulse.

In the blood circulation 16 from the venous cannula 13 to the arterial cannula 14 the blood is enriched with oxygen in the oxygenator 8 and CO₂ is removed.

The blood pump 1 is accelerated by a special value on top of the base speed for a defined period within a maximum time window which is dependent on the current heart frequency. The time limitation takes place by way of a further algorithm.

The blood pump or blood pump 1 and 7 are controlled in such a way that a diastolic augmentation occurs. During this heart action the coronary perfusion pressure is increased. The end-diastolic blood pressure in the area of the aorta close to the heart then falls to a lower value than normal. The following systole has less ejection resistance to overcome and is therefore known as an “influenced systole”. The lower afterload can be seen in the lower systolic pressure.

By increasing the diastolic pressure the oxygen balance of the heart muscle is improved in two ways: the myocardial oxygen supply is increased by a rise in the coronary perfusion pressure and at the same time the mechanical heart action and thereby the myocardial oxygen consumption are decreased. In this way the preconditions for recovery of the heart are improved.

One problem of oxygenators is clotting, whereby the constituents of the blood are deposited on the gas exchange membrane. In addition, clots can form in areas of the oxygenator where there is little flow. Through the pulsatile flow through the oxygenator the flow in the oxygenator changes, as a result of which the service life of the oxygenator is improved.

Furthermore, as a side effect the gas exchange is improved as the boundary layer between fibres and the flowing blood is reduced.

1. Arrangement with a blood pump (1) and pump control unit (2), which has a computer (3) that converts a control signal (4) into a pump actuating signal (5), wherein the pump actuating signal (5) produces a wave-like surging and subsiding pump output for a pulsatile flow.

2. Arrangement according to claim 1, wherein the blood pump (1) also provides a constant basic output.

3. Arrangement according to claim 1, wherein the arrangement has a further blood pump (7) which provides a constant basic output.

4. Arrangement according to claim 1, wherein the arrangement has a further blood pump (7) which provides a wave-like surging and subsiding pump output.

5. Arrangement according to claim 1, further comprising an oxygenator (8) which is supplied by the blood pump (1).

6. Arrangement according to claim 5, wherein in the direction of flow the blood pump (1) is arranged upstream of the oxygenator (8) and a further blood pump (7) is arranged after the oxygenator (8).

7. Arrangement according to claim 5, wherein the oxygenator (8) has a housing (9) and at least one blood pump (1, 7) is arranged in this housing (9).

8. Arrangement according to claim 1, further comprising at least one non-occlusive blood pump (1) such as, in particular, a diagonal, axial or centrifugal pump.

9. Arrangement according to claim 1, further comprising a clock generator which provides the control signal.

10. Arrangement according to claim 1, further comprising an ECG (10) which provides the control signal (4).

11. Arrangement according to claim 1, further comprising an arterial pressure sensor which provides the control signal (4).

12. Arrangement according to claim 1, further comprising an arterial cannula (14) which is longer than 20 cm.

13. Method of operating a blood pump (1) in which the pump (1) is operated with an iterating output in order to produce a wave-like surging and subsiding pulsatile flow.

14. Method according to claim 13, wherein phase-shifted in relation to the pulsatile flow, a further blood pump (7) produces a wave-like surging and subsiding pump output.

15. Method according to claim 13, wherein a base load overlaps the pulsatile flow of at least one pump (1).

16. Method according to claim 13, wherein with the pump (1) the diastolic pressure is increased.

17. Method according to claim 13, wherein the pump (1) directs a flow to an oxygenator (8).
